(19) United States
(12) Patent Application Publication
(10) Pub. No.: US 2018/0072638 A1
(43) Pub. Date: Mar. 15, 2018

(54) PROCESS FOR PREPARING 1,3-BUTADIENE FROM N-BUTENES BY OXIDATIVE DEHYDROGENATION

(71) Applicants: BASF SE, Ludwigshafen (DE); Linde AG, Munich (DE)

(72) Inventors: Jan Pablo JOSCH, Neustadt (DE); Stephan DEUBLEIN, Harthausen (DE); Regina BENFER, Altrip (DE); Friedemann GAITZSCH, Limburgerhof (DE); Hendrik REYNEKE, Munich (DE); Christine TOEGEL, Neubiberg (DE); Ulrike WENNING, Pullach (DE); Anton WELLENHOFER, Hohenschaftlarn (DE); Heinz BOELT, Wolfratshausen (DE)

(21) Appl. No.: 15/561,623

(22) PCT Filed: Mar. 14, 2016
(86) PCT No.: PCT/EP2016/055408

§ 371 Date: Sep. 26, 2017

(30) Foreign Application Priority Data
Mar. 26, 2015 (EP) 15161031.8

(51) Int. Cl. C07C 5/48; C07C 7/11; C07C 7/09; C07C 7/08; C07C 7/00; C07C 11/167

(52) U.S. Cl. CPC C07C 5/48; C07C 7/11; C07C 11/167; C07C 7/08; C07C 7/005; C07C 7/09

(57) ABSTRACT

The invention relates to a process for producing butadiene from n-butenes, comprising the steps of:
A) providing an n-butenes-comprising input gas stream a1,
B) feeding the n-butenes-comprising input gas stream al, an oxygenous gas and an oxygenous cycle gas stream a2 into at least one oxidative dehydrogenation zone and oxidatively dehydrogenating n-butenes to butadiene to obtain a product gas stream b comprising butadiene, unconverted n-butenes, steam, oxygen, low-boiling hydrocarbons, high-boiling secondary components, possibly carbon oxides and possibly inert gases,
Ca) cooling down the product gas stream b and optionally at least partially removing high-boiling secondary components and steam to obtain a product gas stream b′,
Cb) compressing and cooling the product gas stream b′ in at least one compression and cooling stage to obtain at least one aqueous condensate stream c1 and one gas stream c2 comprising butadiene, n-butenes, steam, oxygen, low-boiling hydrocarbons, possibly carbon oxides and possibly inert gases,
Da) absorbing the C₄ hydrocarbons comprising butadiene and n-butenes into an aromatic hydrocarbon solvent absorption medium stream A1 in an absorption column K1 and removing noncondensable and low-boiling gas constituents comprising steam, oxygen, low-boiling hydrocarbons, possibly carbon oxides, aromatic hydrocarbon solvent and possibly inert gases as gas stream d2 from the gas stream c2 to obtain a C₄ hydrocarbons-laden absorption medium stream A1′ and the gas stream d2 and subsequently desorbing the C₄ hydrocarbons from the laden absorption medium stream A1′ to obtain a C₄ product gas stream d1,
Db) at least partially recycling the gas stream d2 into the oxidative dehydrogenation zone as cycle gas stream a2,
wherein said process comprises limiting the content of aromatic hydrocarbon solvent in the cycle gas stream a2 to less than 1 vol % by contacting in a further column K2 the gas stream d2 exiting the removal stage Da) with an at least partially recirculating liquid absorption medium stream A2 for the aromatic hydrocarbon solvent A1, and limiting the water content of the liquid absorption medium stream A2 in the column K2 to no more than 80 wt %.

The invention relates to a process for producing 1,3-butadiene from n-butenes by oxidative dehydrogenation (ODH).

Butadiene (1,3-butadiene) is an important commodity chemical and is used, for example, for producing synthetic rubbers (butadiene homopolymers, styrene-butadiene rubber or nitrile rubber) or for producing thermoplastic terpolymers (acrylonitrile-butadiene-styrene copolymers). Butadiene is also converted into sulfolane, chloroprene and 1,4-hexamethylenediamine (via 1,4-dichlorobutene and adiponitrile). Via dimerization of butadiene, it is further possible to prepare vinylcyclohexene, which may be dehydrogenated to styrene.

Butadiene may be produced by thermal cracking (steam cracking) of saturated hydrocarbons, typically proceeding from naphtha as feedstock. Steam cracking of naphtha generates a hydrocarbon mixture of methane, ethane, ethene, acetylene, propane, propene, propyne, allene, butanes, butenes, butadiene, butynes, methylallene, and C5 and higher hydrocarbons.

Butadiene may also be obtained by oxidative dehydrogenation of n-butenes (1-butene and/or 2-butene) in the presence of molecular oxygen. The input gas stream used for oxidative dehydrogenation (oxydehydrogenation, ODH) of n-butenes to butadiene may be any desired mixture comprising n-butenes. For example, it is possible to use a fraction which comprises n-butenes (1-butene and/or 2-butene) as the main constituent and has been obtained from the C₄ fraction from a naphtha cracker by removing butadiene and isobutene. It is further also possible to use as the input gas stream gas mixtures which comprise 1-butene, cis-2-butene, trans-2-butene or mixtures thereof and which have been obtained by dimerization of ethylene. Further streams usable as the input gas stream are n-butenes-comprising gas mixtures obtained by fluid catalytic cracking (FCC).

As well as n-butenes and molecular oxygen, the reaction gas mixture generally comprises inert components. Here, “inert components” is to be understood as meaning that said components undergo less than 90% conversion under the ODH reaction conditions. Examples of inert components include steam and nitrogen, but also alkanes such as methane. Here, the molar ratio of the inert component to molecular oxygen is generally higher than for air, primarily to avoid the risk of explosions. This may be achieved, for example, by using air as oxygenous gas and diluting it with molecular nitrogen. However, the provision of large volumes of concentrated nitrogen is costly and, from an economic point of view, disadvantageous. This may further be achieved by using molecular oxygen-depleted air (lean air) as the oxygenous gas. This may further be achieved by diluting air with lean air.

Processes for oxidative dehydrogenation of butenes to butadiene are known in principle,

US 2012/0130137A1, for example, describes such a process using catalysts comprising oxides of molybdenum, of bismuth and generally of further metals. In order for such catalysts for oxidative dehydrogenation to exhibit lasting activity, a critical minimum partial oxygen pressure in the gas atmosphere is required to avoid excessive reduction and hence loss of performance of the catalysts. It is thus generally also not possible to operate with stoichiometric oxygen input or complete oxygen conversion in the oxydehydrogenation reactor (ODH reactor). US 2012/0130137 describes, for example, a starting gas oxygen content of from 2,5 to 8 vol %. The N₂/O₂ ratio in the reaction gas mixture is set to the desired value by diluting air as the oxygenous gas with nitrogen gas.

The need for an oxygen excess for such catalyst systems is common knowledge and is reflected in the process conditions when such catalysts are used. Cited here as a representative example are the more recent publications of Jung et al. (Catal. Surv. Asia 2009, 13, 78-93; DOI 10.1007/s10563-009-9069-5 and Applied Catalysis A: General 2007, 317, 244-249; DOI 10.1016/j.apcata.2006.10.021).

However, the presence of oxygen alongside butadiene downstream of the ODH reactor stage in the work-up section of such processes operated with an oxygen excess presents hazards. Particularly in the liquid phase, the formation and accumulation of organic peroxides should be monitored. These hazards are discussed, for example, by D. S. Alexander (Industrial and Engineering Chemistry 1959, 51, 733-738).

JP 2011-006381 A filed by Mitsubishi addresses the hazard of peroxide formation in the work-up section of a process for producing conjugated alkadienes. Presented as a solution thereto is the addition of polymerization inhibitors to the absorption solutions for the process gases and the setting of a maximum peroxide content of 100 wppm by heating the absorption solutions. However avoidance or monitoring of peroxides in upstream process steps is not addressed. A particularly critical aspect is the step of cooling the ODH reactor output with a water quench. Organic peroxides formed are barely soluble in water and accordingly are deposited and may accumulate in the apparatus in solid or liquid form instead of being discharged with the aqueous purge stream from the quench. At the same time, the temperature of the water quench is not high enough that sufficiently high and constant breakdown of the peroxides formed can be assumed.

Catalytic oxidative dehydrogenation can form high-boiling secondary components, for example maleic anhydride, phthalic anhydride, benzaldehyde, benzoic acid, ethylbenzene, styrene, fluorenone, anthraquinone and others. Such deposits can result in blockages and an increased pressure drop in the reactor or downstream of the reactor in the work-up region and can thus disrupt controlled operation. Deposits of the cited high-boiling secondary components can also impair the function of heat exchangers or damage apparatuses with moving parts such as compressors. Steam-volatile compounds such as fluorenone may advance through a water-operated quench apparatus and precipitate downstream thereof in the gas discharge lines. There is thus also a general danger of solid deposits finding their way into downstream apparatus sections, for example compressors, and causing damage thereto.

US 2012/0130137A1 also makes reference to the problem of high-boiling by-products. Particular mention is made of phthalic anhydride, anthraquinone and fluorenone, it being reported that said by-products are typically present in the product gas in concentrations of from 0.001 to 0.10 vol %. Paragraphs [0124]-[0126] of US 2012/0130137A1 recommend cooling down the hot reactor output gases directly by contact with a cooling liquid (quench tower), typically to an initial temperature of from 5° C. to 100° C. Cited cooling liquids are water and aqueous alkali solutions. There is explicit mention of the problem of blockages in the quench due to high boilers from the product gas or due to polymerization products of high-boiling by-products from the product gas and it is thus reported to be advantageous that high-boiling by-products are entrained as little as possible from the reaction section to the cooling section (quench).

KR 2013-0036467 and KR 2013-0036468 likewise recommend cooling down the hot reactor output gases directly by contact with a coolant. Coolants employed are water-soluble organic coolants in order that the secondary components may be better cooled down.

JP 2011-001341A describes two-stage cooling for a process for oxidative dehydrogenation of alkenes to conjugated alkadienes. This comprises first adjusting the temperature of the product output gas from the oxidative dehydrogenation to between 300° C. and 221° C. and then cooling down said gas further to a temperature between 99° C. and 21° C. Paragraphs [0066] ff. state that the adjustment of the temperature to between 300° C. and 221° C. is preferably effected using heat exchangers, but a portion of the high boilers from the product gas could also precipitate-out in these heat exchangers. JP 2011-001341A therefore describes occasional rinsing of deposits out of the heat exchangers with organic or aqueous solvents. Solvents described are, for example, aromatic hydrocarbons such as toluene or xylene, or an alkaline aqueous solvent, for example the aqueous solution of sodium hydroxide. To avoid excessively frequent shutdown of the process to clean the heat exchanger, JP2011-001341A describes a setup having two parallel heat exchangers each alternately operated or purged (referred to as A/B mode).

JP 2010-90083 A describes a process for oxidative dehydrogenation of n-butenes to butadiene where the product gas from the oxidative dehydrogenation is cooled down and dewatered. Butadiene and unconverted butenes and butane are subsequently absorbed from the C₄ hydrocarbons—comprising input gas stream into a solvent. The residual gas not absorbed by the solvent is subsequently sent for disposal by incineration. If a low-boiling solvent such as toluene is used as the absorption medium, said solvent is recovered from the residual gas stream by absorption in a high-boiling solvent, for example decane, to avoid solvent losses.

The residual gas not absorbed by the solvent and largely freed of the C₄ hydrocarbons may also be recycled into the oxydehydrogenation as cycle gas.

JP 2012-072086 A describes that a gas where the hydrocarbons, such as butadiene, n-butene, n-butane and isobutane, have been removed from the product gas mixture can be recycled into the oxydehydrogenation as oxygenous gas. No mention is made of how such a recycle gas stream is obtained nor of which impurities are present therein.

JP 2012-240963 describes a process for butadiene production where the dehydrogenation product gas stream comprising C₄ hydrocarbons is contacted with a first absorbent for the C₄ hydrocarbons in a first absorption stage. In a second absorption stage, the gas stream freed of C₄ hydrocarbons is subsequently contacted with a second liquid absorbent to reduce the content of vaporous first absorbent in the gas stream. This second absorbent has a higher boiling point than the first absorbent. The first absorbent is toluene for example and the second absorbent is a different hydrocarbon having a higher boiling point. The disadvantage is that the first and second absorbent need to be separated from one another for regeneration.

It is an object of the present invention to provide a process which remedies the abovementioned disadvantages of prior art processes. It is a particular object of the present invention to provide a process where deposits due to high-boiling organic secondary constituents in the apparatuses connected downstream of the ODH are avoided. It is a further object of the present invention to provide a process where the potential accumulation of organic peroxides is avoided. It is a further object of the invention to avoid high levels of contamination of wastewater with organic compounds in dissolved, emulsified or suspended form, and to reduce the generation of wastewater contaminated with organic compounds. These objects are to be achieved without severe impairment of catalyst activity by traces of organic solvents in the cycle gas recycled into the ODH.

The object is achieved by a process for producing butadiene from n-butenes, comprising the steps of:
A) providing an n-butenes-comprising input gas stream a1,
B) feeding the n-butenes-comprising input gas stream a1, an oxygenous gas and an oxygenous cycle gas stream a2 into at least one oxidative dehydrogenation zone and oxidatively dehydrogenating n-butenes to butadiene to obtain a product gas stream b comprising butadiene, unconverted n-butenes, steam, oxygen, low-boiling hydrocarbons, high-boiling secondary components, possibly carbon oxides and possibly inert gases,
Ca) cooling down the product gas stream b and optionally at least partially removing high-boiling secondary components and steam to obtain a product gas stream b′,
Cb) compressing and cooling the product gas stream b′ in at least one compression and cooling stage to obtain at least one aqueous condensate stream c1 and one gas stream c2 comprising butadiene, n-butenes, steam, oxygen, low-boiling hydrocarbons, possibly carbon oxides and possibly inert gases,
Da) absorbing the C₄ hydrocarbons comprising butadiene and n-butenes into an aromatic hydrocarbon solvent absorption medium stream Al in an absorption column K1 and removing noncondensable and low-boiling gas constituents comprising steam, oxygen, low-boiling hydrocarbons, possibly carbon oxides, aromatic hydrocarbon solvent and possibly inert gases as gas stream d2 from the gas stream c2 to obtain a C₄ hydrocarbons-laden absorption medium stream A1′ and the gas stream d2 and subsequently desorbing the C₄ hydrocarbons from the laden absorption medium stream A1′ to obtain a C₄ product gas stream d1,
Db) at least partially recycling the gas stream d2 into the oxidative dehydrogenation zone as cycle gas stream a2,
   wherein said process comprises limiting the content of aromatic hydrocarbon solvent in the cycle gas stream a2 to less than 1 vol % by contacting in a further column K2 the gas stream d2 exiting the removal stage Da) with an at least partially recirculating liquid absorption medium stream A2 for the aromatic hydrocarbon solvent A1, and limiting the water content of the liquid absorption medium stream A2 in the further column K2 to no more than 80 wt %.

The volume fractions of the aromatic hydrocarbon solvent and of the further gas constituents are determined by gas chromatography. Calibration for the aromatic hydrocarbon solvent, for example mesitylene, is performed using an external standard. To this end, a gasifiable solvent, for example m-xylene, is dissolved together with mesitylene in a particular molar ratio in a solvent, for example acetone. The mole fraction is converted to parts by volume under the assumption that both substances and the solvent behave as ideal gases.

The gas sample with a known volume fraction of the gasifiable solvent is supplied to the GC via a sample loop. The sample loop of defined volume is operated at constant pressure and constant temperature, an external factor then being determinable from the areas for the comparative substance and the mesitylene for example. Said external factor may then be expressed in terms of mesitylene.

The further components are calibrated individually or in mixtures in a similar manner. This comprises treating all components as ideal gases. This applies equally to the analysis of the gas streams in the ODH process.

It has been found that elevated amounts of aromatic hydrocarbon solvents in the oxydehydrogenation reaction gas mixture impair catalyst activity. The amount of aromatic hydrocarbon solvent in the reaction gas mixture depends on the fraction of aromatic hydrocarbon solvent in the cycle gas and on the fraction of cycle gas in the reaction gas mixture.

The provision of a further column K2 limits the content of aromatic hydrocarbon solvent in the cycle gas stream a2 to less than 1 vol % by absorption of the absorption medium A1 present in vaporous form or in the form of fine droplets in the gas stream d2) downstream of the absorption stage Da) into an absorption medium stream A2. Here, A2 may comprise an absorption medium distinct from A1 or else it may comprise the same absorption medium. Stream A2 may also have a lower temperature than gas stream d2. When the absorption medium present in stream A2 and the absorption medium present in stream A1 are the same, the temperature of stream A2 is actually lower than the temperature of stream d2, the content of aromatic hydrocarbon in stream d2 thus being reduced.

Limiting the water content of this further absorption medium stream A2 in the column K2 to no more than 80 wt % and preferably no more than 50 wt % avoids the solubility of the absorption medium A2 for organic peroxides (primarily butadiene peroxide) being lowered to an extent such that a separate phase of organic peroxides may be formed. The formation of a peroxide phase is a safety issue and it is imperative that said formation be avoided.

The water content of the further absorption medium stream A2 is limited to no more than 80% by weight, preferably no more than 50% by weight.

The choice of coolant in the cooling-down stage Ca) is not subject to any restrictions. However, preference is given to using an organic solvent in the cooling-down stage Ca). These organic solvents generally have a very much higher dissolution capacity for the high-boiling by-products which can lead to deposits and blockages in the plant parts downstream of the ODH reactor than do water or aqueous alkaline solutions. Preferred organic solvents used as cooling agent are aromatic hydrocarbons, particular preference being given to toluene, o-xylene, m-xylene, p-xylene, mesitylene, all possible constitutional isomers of mono-, di- and triethylbenzene and all possible constitutional isomers of mono-, di- and triisopropylbenzene, or mixtures thereof. Preference is given to aromatic hydrocarbons having a boiling point of more than 120° C. at 1013.25 hPa, or mixtures thereof. Mesitylene is specifically preferred.

The absorption medium used in the removal stage Da) is an aromatic hydrocarbon solvent. Preference is given to toluene, o-xylene, m-xylene, p-xylene, mesitylene, all possible constitutional isomers of mono-, di- and triethylbenzene and all possible constitutional isomers of mono-, di- and triisopropylbenzene, or mixtures thereof. Preference is given to aromatic hydrocarbons having a boiling point of more than 120° C. at 1013.25 hPa. Mesitylene is particularly preferred. In particular, the removal stage Da) employs the same aromatic hydrocarbon solvent as the preceding cooling-down stage Ca) when an organic solvent is used in the cooling-down stage Ca).

Absorption of the C₄ hydrocarbons comprising butadiene and n-butenes from the gas stream c2 into the aromatic hydrocarbon solvent A1 affords uncondensable and low-boiling gas constituents comprising steam, oxygen, low-boiling hydrocarbons, possibly carbon oxides and possibly inert gases as gas stream d2. At least some of this gas stream d2 is recycled into the oxidative dehydrogenation (step B)) as cycle gas stream a2. In accordance with the invention the content of aromatic hydrocarbon solvent in the cycle gas stream a2 is less than 1 vol %.

In accordance with the invention the content of aromatic hydrocarbon solvent A1 in the cycle gas stream a2 is limited to less than 1 vol % by contacting in a further column K2 the gas stream d2 exiting the removal stage Da) with a liquid absorption medium A2 for the aromatic hydrocarbon solvent A1. The absorption medium A2 used in this further column K2 needs to be miscible with the aromatic hydrocarbon solvent A1 from the absorption column K1 of the removal stage Da) and may optionally also be the same solvent. When the absorption medium A2 used in the further column K2 is the same solvent as the absorption medium A1 used in the column K1, the pressure in this further column K2 is higher than in the absorption column K1 of the removal stage Da) or else the absorption medium stream A2 supplied to this further column K2 is cooler than the gas stream d2 entering this column and the aromatic hydrocarbon solvent A1 present in the gas stream d2 is thus at least partially removed from the gas stream d2.

Also in accordance with the invention the water content of the absorption medium A2 in the further column K2 is limited to no more than 80 wt %, preferably no more than 50 wt %. This may be achieved by
(i) continually withdrawing some of the water-containing absorption medium A2 from the further column K2 and replacing it with fresh absorption medium A2 containing no water or less water; or
(ii) separating the water-containing absorption medium into an absorption medium phase and a water phase in a phase separator, removing the water phase and reintroducing the absorption medium phase into the further column K2; the phase separator may be a separate phase separator or it may be an integral part of the column bottom of the further column K2; or
(iii) passing some of the water-containing absorption medium into the absorption column K1.

In one embodiment of the invention the absorption column K1 used in the absorption step Da) or the further column K2 used downstream of the absorption step Da) comprises one or more apparatuses, for example a demister or droplet separator, which reduce entrainment of liquid constituents from the absorption column K1 or the further column K2 into the gas stream d2. Suitable apparatuses are all apparatuses which reduce the fraction of liquid constituents in the gas stream d2. In general, demisters or droplet separators are understood to mean apparatuses for separating ultrafine liquid droplets from gases, vapors or mists, generally aerosols. In columns, liquid entrainment may be reduced via demisters or droplet separators. Demisters or droplet separators may be made of, for example, wire knit packings, lamellar separators or beds of random packings having a high internal surface area. The materials of construction employed are generally steels, chromium-nickel steels, aluminum, copper, nickel, polypropylene, polytetra-fluoroethylene and the like. The separation level decreases with decreasing droplet diameters. Demisters may be counted among the coalescence separators. Demisters are described, inter alia, in applications U.S. Pat. No. 3,890,123 and U.S. Pat. No. 4,141,706 and the documents cited therein. The demister or droplet separator may be disposed either inside the absorption column or absorption columns, or be connected downstream thereof.

The aromatic hydrocarbon solvent content of the cycle gas stream a2 is preferably less than 0.5 vol %, more preferably less than 0.2 vol %, in particular less than 0.1 vol %.

It is preferable when the process according the invention further comprises the following additional process steps:

E) separating the C₄ product stream d1 by extractive distillation with a butadiene-selective solvent into a stream e1 comprising butadiene and the selective solvent and a stream e2 comprising n-butenes;

F) distilling the stream e2 comprising butadiene and the selective solvent to obtain a stream f1 comprising the selective solvent and a stream f2 comprising butadiene.

Embodiments which follow are preferred or particularly preferred versions of the process according to the invention:

At least one cooling stage in which the product gas stream b is cooled by indirect cooling in a heat exchanger may be provided upstream of stage Ca).

Stage Ca) may be performed in a plurality of stages Ca1) to Can), preferably in two stages Ca1) and Ca2). Here, particular preference is given to supplying at least some of the coolant, as a cooling agent, to the first stage Ca1) after it has passed through the second stage Ca2).

Stage Cb) generally comprises at least one compression stage Cba) and at least one cooling-down stage Cbb). It is preferable when in the at least one cooling-down stage Cbb) the gas compressed in the compression stage Cba) is contacted with a cooling agent. It is more preferable when the cooling agent of the cooling-down stage Cbb) comprises the same organic solvent used as cooling agent in stage Ca) when an organic solvent is used in the cooling-down stage Ca). In a particularly preferred version, at least some of this cooling agent is supplied as cooling agent to stage Ca) after it has passed through the at least one cooling-down stage Cbb) The cooling-down stage Cbb) may alternatively consist of heat exchangers.

It is preferable when stage Cb) comprises a plurality of compression stages Cba1) to Cban) and cooling-down stages Cbb1) to Cbbn), for example four compression stages Cba1) to Cba4) and four cooling-down stages Cbb1) to Cbb4).

It is preferable when step Da) comprises steps Daa) to Dac);
Daa) absorbing the C₄ hydrocarbons comprising butadiene and n-butenes into an aromatic hydrocarbon solvent as absorption medium to obtain a C₄ hydrocarbons-laden absorption medium stream and the gas stream d2,
Dab) removing oxygen from the C₄ hydrocarbons-laden absorption medium stream from step Daa) by stripping with a noncondensable gas stream, and
Dac) desorbing the C₄ hydrocarbons from the laden absorption medium stream to obtain a C₄ product gas stream d1 consisting essentially of C₄ hydrocarbons and comprising less than 100 ppm of oxygen.

Embodiments of the process according to the invention are described in detail below:

The input gas streams a1 employed may be pure n-butenes (1-butene and/or cis-2-butene and/or trans-2-butene) but also gas mixtures comprising butenes. Such a gas mixture may be obtained, for example, by nonoxidative dehydrogenation of n-butane. It is also possible to use a fraction which comprises n-butenes as the main constituent and has been obtained from the C₄ fraction from naphtha cracking by removal of butadiene and isobutene. It is further also possible to use gas mixtures as input gas stream which comprise pure 1-butene, cis-2-butene, trans-2-butene or mixtures thereof and which have been obtained by dimerization of ethylene. Further streams usable as the input gas stream are n-butenes-comprising gas mixtures obtained by fluid catalytic cracking (FCC).

In one embodiment of the process according to the invention the n-butenes-comprising input gas stream is obtained by nonoxidative dehydrogenation of n-butane. The coupling of a nonoxidative catalytic dehydrogenation with oxidative dehydrogenation of the n-butenes formed makes it is possible to obtain a high yield of butadiene based on the n-butane employed. Nonoxidative catalytic n-butane dehydrogenation affords a gas mixture comprising not only butadiene, 1-butene, 2-butene and unconverted n-butane but also secondary constituents. Typical secondary constituents are hydrogen, steam, nitrogen, CO and CO₂, methane, ethane, ethene, propane and propene. The composition of the gas mixture exiting the first dehydrogenation zone may vary significantly depending on the mode of operation of the dehydrogenation. For instance, when dehydrogenation is performed while feeding oxygen and additional hydrogen, the product gas mixture has a comparatively high content of steam and carbon oxides. For modes of operation without oxygen feeding, the product gas mixture of the nonoxidative dehydrogenation has a comparatively high content of hydrogen.

Step B) comprises feeding the reaction gas mixture comprising the n-butenes-comprising input gas stream a1, an oxygenous gas, an oxygenous cycle gas stream a2 and optionally further components into at least one dehydrogenation zone (the ODH reactor) and oxidatively dehydrogenating the butenes present in the gas mixture to butadiene in the presence of an oxydehydrogenation catalyst.

Catalysts suitable for the oxydehydrogenation are generally based on an Mo—Bi—O-containing multimetal oxide system which generally additionally comprises iron. In general, the catalyst system also comprises further additional components, for example potassium, cesium, magnesium, zirconium, chromium, nickel, cobalt, cadmium, tin, lead, germanium, lanthanum, manganese, tungsten, phosphorus, cerium, aluminum or silicon. Iron-containing ferrites too have been proposed as catalysts.

In one preferred embodiment, the multimetal oxide comprises cobalt and/or nickel. In a further preferred embodiment, the multimetal oxide comprises chromium. In a further preferred embodiment, the multimetal oxide comprises manganese.

Examples of Mo—Bi—Fe—O-containing multimetal oxides are Mo—Bi—Fe—Cr—O— or Mo—Bi—Fe—Zr—O-containing multimetal oxides. Preferred systems are described, for example, in U.S. Pat. No. 4,547,615 (Mo₁₂BiFe_{0.1}Ni₈ZrCr₃K_{0.2}Oₓ and Mo₁₂BiFe_{0.1}Ni₈AlCr₃K_{0.2}Oₓ), U.S. Pat. No. 4,424,141 (Mo₁₂BiFe₃Co_{4.5}Ni_{2.5}P_{0.5}K_{0.1}Oₓ+SiO₂), DE-A 25 30 959 (Mo₁₂BiFe₃Co_{4.5}Ni_{2.5}Cr_{0.5}K_{0.1}Oₓ, Mo_{13.75}BiFe₃Co_{4.5}Ni_{2.5}Ge_{0.5}K_{0.8}Oₓ, Mo₁₂BiFe₃Co_{4.5}Ni_{2.5}Mn_{0.5}K_{0.1}Oₓ and Mo₁₂BiFe₃Co_{4.5}Ni_{2.5}La_{0.5}K_{0.1}Oₓ), U.S. Pat. No. 3,911,039 (Mo₁₂BiFe₃Co_{4.5}Ni_{2.5}Sn_{0.5}K_{0.1}Oₓ), DE-A 25 30 959 and DE-A 24 47 825 (Mo₁₂BiFe₃Co_{4.5}Ni_{2.5}W_{0.5}K_{0.1}Oₓ).

Furthermore, suitable multimetal oxides and the production thereof are described in U.S. Pat. No. 4,423,281 (Mo₁₂BiNi₈Pb_{0.5}Cr₃K_{0.2}Oₓ and Mo₁₂Bi_{b}Ni₇Al₃Cr_{0.5}K_{0.5}Oₓ), U.S. Pat. No. 4,336,409 (Mo₁₂BiNi₆Cd₂Cr₃P_{0.5}Oₓ), DE-A 26 00 128 (Mo₁₂BiNi_{0.5}Cr₃P_{0.5}Mg_{7.5}K_{0.1}Oₓ+SiO₂) and DE-A 24 40 329 (Mo₁₂BiCo_{4.5}Ni_{2.5}Cr₃P_{0.5}K_{0.1}Oₓ).

Particularly preferred catalytically active multimetal oxides comprising molybdenum and at least one further metal have the general formula (Ia):

Mo₁₂BiₐFe_{b}Co_{c}Ni_{d}CrₑX¹_{f}X²_{g}O_{y} (Ia),

where
X¹=Si, Mn and/or Al,
X²=Li, Na, K, Cs and/or Rb,
0.2≦a≦1,
0.5 5≦b≦10,
0.5≦c≦10,
0.5≦d≦10,
2≦c+d≦10
0≦e≦2,
0≦f≦10,
0≦g≦0.5,
y=a number which, with the prerequisite of charge neutrality, is determined by the valency and prevalence of the elements other than oxygen in (Ia).

Preference is given to catalysts whose catalytically active oxide composition, of the two metals Co and Ni, comprises only Co (d=0). X¹ is preferably Si and/or Mn and X² is preferably K, Na and/or Cs, it being particularly preferred when X²=K.

The gas comprising molecular oxygen generally comprises more than 10 vol %, preferably more than 15 vol % and even more preferably more than 20 vol % of molecular oxygen. Said gas is preferably air. The upper limit for the content of molecular oxygen is generally no more than 50 vol %, preferably no more than 30 vol % and even more preferably no more than 25 vol %. The gas comprising molecular oxygen may further comprise any desired inert gases. Examples of possible inert gases include nitrogen, argon, neon, helium, CO, CO₂ and water. For nitrogen, the amount of inert gases is generally no more than 90 vol %, preferably no more than 85 vol % and even more preferably no more than 80 vol %. In the case of constituents other than nitrogen, said amount is generally no more than 10 vol %, preferably no more than 1 vol %.

To carry out the oxidative dehydrogenation at full conversion of n-butenes, preference is given to a gas mixture having a molar oxygen:n-butenes ratio of at least 0.5. Preference is given to operating with an oxygen:n-butenes ratio of from 0.55 to 10. This value may be adjusted by mixing the input gas stream with oxygen or at least one oxygenous gas, for example air, and optionally additional inert gas or steam. The oxygenous gas mixture obtained is then supplied to the oxydehydrogenation.

Additionally, the reaction gas mixture may further comprise inert gases such as nitrogen and also water (as steam). Nitrogen may serve to adjust the oxygen concentration and to prevent the formation of an explosive gas mixture, the same applying for steam. Steam further serves to control coking of the catalyst and to remove reaction heat.

The reaction temperature of the oxydehydrogenation is generally controlled by a heat transfer medium surrounding the reaction tubes. Examples of suitable liquid heat transfer media of this type include melts of salts such as potassium nitrate, potassium nitrite, sodium nitrite and/or sodium nitrate, and melts of metals such as sodium, mercury and alloys of various metals. However, ionic liquids or heat-transfer oils may also be used. The temperature of the heat transfer medium is between 220° C. to 490° C., preferably between 300° C. to 450° C. and more preferably between 350° C. and 420° C.

A consequence of the exothermicity of the reactions taking place is that, during the reaction, the temperature in certain sections of the reactor interior may be higher than the temperature of the heat transfer medium, thus leading to hotspot formation. The position and magnitude of the hotspot is determined by the reaction conditions but may also be regulated via the dilution ratio of the catalyst layer or the flow rate of mixed gas. The temperature difference between a hotspot and the heat transfer medium is generally between 1° C. to 150° C., preferably between 10° C. to 100° C. and more preferably between 20° C. to 80° C. The temperature at the end of the catalyst bed is generally between 0° C. to 100° C., preferably between 0.1° C. to 50° C., more preferably between 1° C. to 25° C. higher than the temperature of the heat transfer medium.

The oxydehydrogenation may be performed in any prior art fixed bed reactor, for example in a staged oven, in a fixed bed tubular reactor or shell and tube reactor, or in a plate heat exchanger reactor. A shell and tube reactor is preferred.

The oxidative dehydrogenation is preferably performed in fixed bed tubular reactors or fixed bed shell and tube reactors. The reaction tubes (similarly to the other elements of the shell and tube reactor) are generally manufactured from steel. The wall thickness of the reaction tubes is typically from 1 to 3 mm. The internal diameter thereof is generally (uniformly) from 10 to 50 mm or from 15 to 40 mm, often from 20 to 30 mm. The number of reaction tubes accommodated in a shell and tube reactor generally totals at least 1000, or 3000, or 5000, preferably at least 10 000. The number of reaction tubes accommodated in the shell and tube reactor is often from 15 000 to 30 000, or to 40 000 or to 50 000. The length of the reaction tubes normally extends to just a few meters, a typical reaction tube length being in the range of from 1 to 8 m, often from 2 to 7 m, in many cases from 2.5 to 6 m.

Furthermore, the catalyst layer provided in the ODH reactor may consist of a single layer or of 2 or more layers. These layers may consist of pure catalyst or may be diluted with a material reactive toward neither the input gas stream nor components of the product gas from the reaction. Furthermore, the catalyst layers may consist of all-active material or supported coated catalysts.

The product gas stream exiting the oxidative dehydrogenation comprises not only butadiene but generally also unconverted 1-butene and 2-butene, oxygen and steam. Generally, said stream further comprises as secondary components carbon monoxide, carbon dioxide, inert gases (principally nitrogen), low-boiling hydrocarbons such as methane, ethane, ethene, propane and propene, butane and isobutane, possibly hydrogen and possibly oxygen-containing hydrocarbons known as oxygenates. Examples of oxygenates include formaldehyde, furan, acetic acid, maleic anhydride, formic acid, methacrolein, methacrylic acid, crotonaldehyde, crotonic acid, propionic acid, acrylic acid, methyl vinyl ketone, styrene, benzaldehyde, benzoic acid, phthalic anhydride, fluorenone, anthraquinone and butyraldehyde.

The product gas stream at the reactor outlet is characterized by a temperature close to the temperature at the end of the catalyst bed. The product gas stream is then brought to a temperature of from 150° C. to 400° C., preferably from 160° C. to 300° C., more preferably from 170° C. to 250° C. It is possible to insulate the line through which the product gas stream flows or to employ a heat exchanger in order to keep the temperature within the desired range. This heat exchanger system may be of any desired type provided that said system can be used to keep the temperature of the product gas at the desired level. Examples of heat exchangers include spiral heat exchangers, plate heat exchangers, double tube heat exchangers, multitube heat exchangers, boiler-spiral heat exchangers, boiler-shell heat exchangers, liquid-liquid contact heat exchangers, air heat exchangers, direct contact heat exchangers and fin tube heat exchangers. Since some of the high-boiling by-products present in the product gas may precipitate out during adjustment of the product gas temperature to the desired temperature, the heat exchanger system should thus preferably comprise two or more heat exchangers. When two or more heat exchangers are arranged in parallel to enable distributed cooling of the obtained product gas in the heat exchangers, the amount of high-boiling by-products deposited in the heat exchangers decreases and the service life thereof can therefore be extended. As an alternative to the above-mentioned method, the two or more heat exchangers provided may be arranged in parallel. The product gas is supplied to one or more, but not to all, heat exchangers, which are relieved by other heat exchangers after a certain operating duration. In this method, cooling can be continued, some of the reaction heat can be recovered and, simultaneously, the high-boiling by-products deposited in one of the heat exchangers can be removed. It is possible to use as an abovementioned organic solvent any solvent provided that it is capable of dissolving the high-boiling by-products. Examples include aromatic hydrocarbon solvents, for example toluene and xylenes, and alkaline aqueous solvents, for example the aqueous solution of sodium hydroxide.

A large proportion of the high-boiling secondary components and of the water is then removed from the product gas stream by cooling-down and compression. This stage is also referred to hereinafter as the quench. This quench may consist of only one stage or of a plurality stages. The cooling-down may be effected by contacting with a coolant, preferably an organic solvent. Media employed as cooling medium are organic solvents, preferably aromatic hydrocarbons, more preferably toluene, o-xylene, m-xylene, p-xylene, mesitylene, all possible constitutional isomers of mono-, di- and triethylbenzene and all possible constitutional isomers of mono-, di- and triisopropylbenzene or mixtures thereof. Preference is further given to aromatic hydrocarbons having a boiling point of more than 120° C. at 1013.25 hPa, or mixtures thereof.

Preference is given to a two-stage quench, i.e. stage Ca) comprises two cooling-down stages Ca1) and Ca2) in which stages the product gas stream b is contacted with the organic solvent.

Depending on the presence and temperature level of a heat exchanger upstream of the quench, the temperature of the product gas is generally from 100° C. to 440° C. The product gas is contacted with the cooling medium in the 1st quench stage. This may comprise introducing the cooling medium via a nozzle in order to achieve the best possible efficiency of commixing with the product gas. The same purpose may be served by introducing internals into the quench stage, for example further nozzles, the product gas and the cooling medium passing therethrough together. The coolant inlet into the quench has a configuration such that blockage due to deposits in the region of the coolant inlet is minimized.

The first quench stage generally cools the product gas to from 5° C. to 180° C., preferably from 30° C. to 130° C. and even more preferably from 60° C. to 110° C. The temperature of the coolant medium at the inlet may generally be from 25° C. to 200° C., preferably from 40° C. to 120° C., more particularly from 50° C. to 90° C. The pressure in the first quench stage is not particularly restricted but is generally from 0.01 to 4 bar (g), preferably from 0.1 to 2 bar (g) and more preferably from 0.2 to 1 bar (g). When the product gas comprises relatively large amounts of high-boiling by-products, polymerization of high-boiling by-products and deposits of solids caused by high-boiling by-products in this process section may readily occur. The quench stage is generally configured as a cooling tower. The cooling medium employed in the cooling tower is often employed in circulating fashion. The circulation flow rate of the cooling medium in liters per hour relative to the mass flow rate of butadiene in grams per hour may generally be from 0.0001 to 5 l/g, preferably from 0.001 to 1 l/g and more preferably from 0.002 to 0.24.

The temperature of the cooling medium at the bottom may generally be from 27° C. to 210° C., preferably from 45° C. to 130° C., more preferably from 55° C. to 95° C. Since the loading of the cooling medium with secondary components increases over time, some of the laden cooling medium may be withdrawn from circulation as a purge stream and the amount circulating may be kept constant by adding unladen cooling medium. The ratio of volume discharged to volume added depends on the vapor loading of the product gas and the product gas temperature at the end of the first quench stage.

Depending on the temperature, pressure and water content of the product gas, condensation of water may occur in the first quench stage. In this case, an additional aqueous phase may form, which may further comprise water-soluble secondary components. This may then be withdrawn at the bottom of the quench stage. Preference is given to a mode of operation in which no aqueous phase forms in the first quench stage.

The cooled-down product gas stream possibly depleted of secondary components may then be supplied to a second quench stage. In this stage, said stream may once again be contacted with a cooling medium.

The choice of coolant is not particularly restricted. Media employed as cooling medium are preferably organic solvents, preferably aromatic hydrocarbons, more preferably toluene, o-xylene, m-xylene, p-xylene, mesitylene, all possible constitutional isomers of mono-, di- and triethylbenzene and all possible constitutional isomers of mono-, di- and triisopropylbenzene, or mixtures thereof. Preference is further given to aromatic hydrocarbons having a boiling point of more than 120° C. at 1013.25 hPa, or mixtures thereof.

The product gas is generally cooled to from 5° C. to 100° C., preferably to from 15° C. to 85° C. and even more preferably to from 30° C. to 70° C. before reaching the gas outlet. The coolant can be supplied in countercurrent to the product gas. In this case, the temperature of the coolant medium at the coolant inlet may be from 5° C. to 100° C., preferably from 15° C. to 85° C., more preferably from 30° C. to 70° C. The pressure in the second quench stage is not particularly restricted, but is generally from 0.01 to 4 bar (g), preferably from 0.1 to 2 bar (g) and more preferably from 0.2 to 1 bar (g). The second quench stage is preferably configured as a cooling tower. The cooling medium used in the cooling tower is often employed in circulating fashion. The circulation flow rate of the cooling medium in liters per hour relative to the mass flow rate of butadiene in grams per hour may generally be from 0.0001 to 5 l/g, preferably from 0.3001 to 1l/g and more preferably from 0.002 to 0.2 l/g.

Depending on the temperature, pressure and water content of the product gas, condensation of water may occur in the second quench stage. In this case, an additional aqueous phase may form, which may further comprise water-soluble secondary components. Said phase may then be withdrawn at the bottom of the quench stage. The temperature of the cooling medium at the bottom may generally be from 20° C. to 210° C., preferably from 35° C. to 120° C., more preferably from 45° C. to 85° C. Since the loading of the cooling medium with secondary components increases over time, some of the laden cooling medium may be withdrawn from circulation as a purge stream and the amount circulating may be kept constant by adding unladen cooling medium.

In order to achieve the best possible contact of product gas and cooling medium, the second quench stage may comprise internals. Examples of such internals include bubble-cap, centrifugal and/or sieve trays, columns comprising structured packings, for example sheet metal packings having a specific surface area of from 100 to 1000 m²/m³ such as Mellapak® 250 Y, and random-packed columns.

The coolant circuits of the two quench stages may either be separate from one another or connected to one another. Thus, for instance, the stream may be supplied to the stream or may replace it. The desired temperature of the circulating streams may be established via suitable heat exchangers.

In one preferred embodiment of the invention the cooling-down stage Ca) is thus performed in two stages, the coolant laden with secondary components from the second stage Ca2) being passed into the first stage Ca1). The coolant withdrawn from the second stage Ca2) comprises a reduced amount of secondary components compared to the coolant withdrawn from the first stage Ca1).

Entrainment of liquid constituents from the quench into the offgas line may be minimized by suitable physical measures, for example installation of a demister. Furthermore, high-boiling substances not separated from the product gas in the quench may be removed from the product gas by further physical measures, for example further gas scrubs.

A gas stream is obtained which comprises n-butane, 1-butene, 2-butenes, butadiene, oxygen, hydrogen, steam, small amounts of methane, ethane, ethene, propane and propene, isobutane, carbon oxides, inert gases and fractions of the coolant employed in the quench. This gas stream may further comprise remaining traces of high-boiling components not removed quantitatively in the quench. Examples of such high-boiling components include methyl vinyl ketone, methyl ethyl ketone, crotonaldehyde, acrylic acid, propionic acid, maleic anhydride, ethylbenzene, styrene, furanone, benzoic acid, benzaldehyde, fluorenone and anthraquinone. This gas stream may further comprise formaldehyde, methacrolein and/or furan.

The gas stream b′ from the cooling-down step Ca) depleted of high-boiling secondary components is subsequently cooled down in step Cb) in at least one compression stage Cba) and preferably in at least one cooling-down stage Cbb).

The product gas stream from the quench is compressed in at least one compression stage and subsequently cooled down further in the cooling apparatus to form at least one condensate stream comprising water. When the quench employs a coolant other than water the coolant used in the quench may further condense out and may possibly form a separate phase. What remains is a gas stream comprising butadiene, 1-butene, 2-butenes, oxygen, steam, possibly low-boiling hydrocarbons such as methane, ethane, ethene, propane and propene, butane and isobutane, possibly carbon oxides and possibly inert gases. This product gas stream may further comprise traces of high-boiling components.

The compression and cooling of the gas stream may be effected in one stage or in a plurality of stages (n stages). Overall, the stream is generally compressed from a pressure in the range from 1.0 to 4.0 bar (absolute) to a pressure in the range from 3.5 to 20 bar (absolute). Each compression stage is followed by a cooling-down stage in which the gas stream is cooled down to a temperature in the range of from 15° C. to 60° C. The cooling-down is preferably effected by contacting with an organic solvent as cooling agent. Alternatively, heat exchangers may be employed. In the case of compression being effected in a plurality of stages, the condensate stream may thus also comprise a plurality of streams. The condensate stream consists largely of water (aqueous phase) and of any coolant used in the quench (organic phase). Both streams (aqueous and organic phase) may additionally comprise, to a small extent, secondary components such as low boilers, C₄ hydrocarbons, oxygenates and carbon oxides.

In order to cool the stream and/or to remove further secondary components from the stream, the condensed quench coolant may be cooled down in a heat exchanger and recycled into the apparatus as coolant. Since the loading of this cooling medium with secondary components increases over time, some of the laden cooling medium may be withdrawn from circulation and the amount of the cooling medium circulating may be kept constant by adding unladen coolant.

The coolant added as cooling medium thus likewise preferably consists of the aromatic hydrocarbon solvent used as quench coolant.

The condensate stream may be recycled into the circulation stream of the quench. This makes it possible to return the C₄ components absorbed in the condensate stream to the gas stream and thus to increase the yield. Examples of suitable compressors include turbocompressors, rotary piston compressors and reciprocating piston compressors. The compressors may be driven with an electric motor, an expander, or a gas or steam turbine. Typical compression ratios (exit pressure:entry pressure) per compressor stage are between 1.5 and 3.0 depending on type. The cooling-down of the compressed gas is effected with organic solvent-purged heat exchangers or organic quench stages, which may be shell and tube, spiral or plate heat exchangers for example. Employed in these heat exchangers as coolant are cooling water or heat transfer oils. Preference is additionally given to air cooling using blowers.

The gas stream c2 comprising butadiene, n-butenes, oxygen, low-boiling hydrocarbons (methane, ethane, ethene, propane, propene, n-butane, isobutane), steam, possibly carbon oxides, possibly inert gases and possibly traces of secondary components is sent for further processing as an output stream.

A step Da) comprises removing noncondensable and low-boiling gas constituents comprising steam, oxygen, low-boiling hydrocarbons (methane, ethane, ethene, propane, propene), carbon oxides and inert gases from the process gas stream c2 in an absorption column K1 by absorbing the C₄ hydrocarbons into an aromatic hydrocarbon solvent high-boiling absorption medium A1 and subsequently desorbing the C₄ hydrocarbons. Step Da) preferably comprises the steps Daa) to Dac:

Daa) absorbing the C₄ hydrocarbons comprising butadiene and n-butenes into an aromatic hydrocarbon solvent absorption medium A1 to obtain a C₄ hydrocarbons-laden absorption medium stream A1′ and the gas stream d2,

Dab) removing oxygen from the C₄ hydrocarbons-laden absorption medium stream A1′ from step Daa) by stripping with a noncondensable gas stream, and
Dac) desorbing the C₄ hydrocarbons from the laden absorption medium stream A1′ to obtain a C₄ product gas stream d1 consisting essentially of C₄ hydrocarbons.

To this end, in the absorption stage the gas stream c2 is contacted with the absorption medium A1 and the C₄ hydrocarbons are absorbed into the absorption medium A1 to obtain an absorption medium A1′ laden with C₄ hydrocarbons and a gas stream d2 comprising the remaining gas constituents, said stream d2 being at least partially recycled into the oxidative dehydrogenation as cycle gas stream. The C₄ hydrocarbons are liberated from the laden absorption medium A1′ again in a desorption stage.

Media employed as absorption medium A1 are organic solvents, preferably aromatic hydrocarbons, more preferably toluene, o-xylene, m-xylene, p-xylene, mesitylene, all possible constitutional isomers of mono-, di- and triethylbenzene and all possible constitutional isomers of mono-, di- and triisopropylbenzene, or mixtures thereof. Preference is further given to aromatic hydrocarbons having a boiling point of more than 120° C. at 1013.25 hPa, or mixtures thereof. In particular, the removal stage Da) employs the same aromatic hydrocarbon solvent as the preceding cooling-down stage Ca) when an organic solvent is used in the cooling-down stage Ca). Preferred absorption media are solvents having a dissolution capacity for organic peroxides of at least 1000 ppm (mg of active oxygen/kg of solvent). In one preferred embodiment the absorption medium A1 employed is mesitylene.

The absorption stage may be performed in any desired suitable absorption column known to those skilled in the art. The absorption may be effected by simply passing the product gas stream through the absorption medium. However, said absorption may also be effected in columns or rotary absorbers. Said absorption may be operated in cocurrent, countercurrent or crosscurrent. The absorption is preferably performed in countercurrent. Examples of suitable absorption columns include tray columns comprising bubble caps, centrifugal and/or sieve trays, columns comprising structured packings, for example sheet metal packings having a specific surface area of from 100 m²/m³ to 1000 m²/m³ such as Mellapak® 250 Y, and random-packed columns. Also useful, however, are trickle towers and spray towers, graphite block absorbers, surface absorbers such as thick-film and thin-film absorbers, and also rotary columns, plate scrubbers, cross-spray scrubbers and rotary scrubbers.

The absorption column K1 is preferably a tray column comprising bubble cap, centrifugal and/or sieve trays or a column comprising structured packings or a random-packed column, more preferably a column comprising structured packings. Said column generally comprises from 10 to 40 theoretical plates. The absorption column K1 is generally operated at a pressure of from 5 to 15 bar, preferably from 8 to 12 bar. The temperature of the absorption medium A1 introduced into column K1 is generally from 5° C. to 50° C., preferably from 20° C. to 40° C.

In one embodiment, the lower region of the absorption column K1 is supplied with the gas stream c2 comprising butadiene, n-butenes and the low-boiling and noncondensable gas constituents. The absorption medium is introduced at the top of the absorption column.

Withdrawn at the top of the absorption column K1 is a gas stream d2 comprising essentially steam, oxygen, low-boiling hydrocarbons (methane, ethane, ethene, propane, propene), the aromatic hydrocarbon solvent, possibly C₄ hydrocarbons (butane, butenes, butadiene), possibly inert gases and possibly carbon oxides. In accordance with the invention, this stream is contacted in the further column K2 with a liquid absorption medium A2 for the aromatic hydrocarbon solvent and subsequently at least partially supplied to the ODH reactor as cycle gas stream a2. This makes it possible, for example, to set the feed stream of the ODH reactor to the desired C₄ hydrocarbon content. Generally, optionally after removal of a purge gas stream, at least 30 vol %, preferably at least 50 vol %, of the gas stream d2 is recycled into the oxidative dehydrogenation zone as cycle gas stream a2. The purge gas stream may be subjected to a thermal or catalytic postcombustion. Said stream may in particular be thermally recovered.

The recycle stream is generally from 10 to 70 vol %, preferably from 30 to 60 vol %, based on the sum total of all streams fed into the oxidative dehydrogenation B).

In accordance with the invention, the content of aromatic hydrocarbon solvent in the cycle gas stream a2 is limited to less than 1 vol % by contacting in a further column K2 the gas stream d2 exiting the removal stage Da) with a liquid absorption medium A2 for the aromatic hydrocarbon solvent, the water content of the absorption medium A2 in the further column K2 being limited to no more than 50 wt %. This may be achieved by
(i) continually withdrawing some of the water-containing absorption medium A2 from the further column K2 and replacing it with fresh absorption medium A2 containing no water or less water; or
(ii) separating the water-containing absorption medium into an absorption medium phase and a water phase in a phase separator, removing the water phase and reintroducing the absorption medium phase to the further column; the phase separator may be a separate phase separator or it may be an integral part of the column bottom of the further column; or
(iii) passing some of the water-containing absorption medium A2 from the column K2 into the absorption column K1.

Suitable absorption media are organic solvents, preferably aromatic hydrocarbons, more preferably toluene, o-xylene, m-xylene, p-xylene, mesitylene, all possible constitutional isomers of mono-, di- and triethylbenzene and all possible constitutional isomers of mono-, di- and triisopropylbenzene, or mixtures thereof. Preferred absorption media are solvents having a dissolution capacity for organic peroxides of at least 1000 ppm (mg of active oxygen/kg of solvent). In one particularly preferred embodiment the absorption medium A2 employed is mesitylene. In particular the absorption medium A2 employed in the further column K2 is the same aromatic hydrocarbon solvent also used as absorption medium A1 in the absorption column K1.

Examples of columns suitable for use as further absorption column K2 include tray columns comprising bubble caps, centrifugal and/or sieve trays, columns comprising structured packings, for example sheet metal packings having a specific surface area of from 100 m²/m³ to 1000 m²/m³ such as Mellapak® 250 Y, and random-packed columns. Said columns generally comprise from 1 to 15 theoretical plates. The further column K2 is generally operated at a pressure of from 5 to 15 bar, preferably from 8 to 12 bar. The temperature of the absorption medium A2 introduced into column K2 is generally from 0° C. to 30° C., preferably from 5° C. to 15° C.

When column K1 and column K2 are operated at the same pressure, the absorption medium A2 introduced to the column K2 is generally at a temperature which is from 1° C. to 50° C. and preferably from 20° C. to 30° C. lower than the temperature of the absorption medium A1 introduced to the column K1.

A first version (i) comprises continually withdrawing some of the water-containing absorption medium A2 from the further column K2 and replacing it with fresh absorption medium A2 containing no water or less water. The fraction of the water-containing absorption medium stream A2 withdrawn and not reintroduced into column K2 is generally from 0.1% to 10% of the total stream of the absorption medium A2. The stream withdrawn is passed either into the column K1 or into a solvent regeneration as desired.

A second version (ii) comprises separating the water-containing absorption medium into an absorption medium phase and a water phase in a phase separator, removing the water phase and reintroducing the absorption medium phase into the further column K2. The phase separator may be a separate phase separator or it may be an integral part of the column bottom of the further column K2.

A third version (iii) comprises passing some of the water-containing absorption medium from the column K2 into the absorption column K1. When two separate columns are concerned, some of the bottoms discharge from the column K2 is passed into the column K1. However, it is also possible for some of the stream from column K2 to discharge into the column K1 via an overflow while the remainder of the stream is withdrawn and reintroduced into the top of the column K2, the column K2 and the column K1 being column sections of a single combined column. In one embodiment, this combined column comprises a chimney tray between the column sections K1 and K2. The fraction of the water-containing absorption medium A2 passed into absorption column K1 and not reintroduced into column K2 is generally from 0.1 to 10% of the total stream of the absorption medium stream A2.

At the bottom of the absorption column K1 in a further column K3, residues of oxygen dissolved in the absorption medium may be discharged by purging with a gas. The fraction of oxygen remaining is preferably sufficiently small that the stream d1 which comprises butane, butenes and butadiene and exits the desorption column comprises only no more than 100 ppm of oxygen.

The stripping-out of the oxygen in step Dab) may be performed in any desired suitable column known to those skilled in the art. The stripping may be effected simply by passing noncondensable gases, preferably inert gases such as nitrogen, through the laden absorption solution. C₄ hydrocarbons stripped out at the same time are scrubbed back into the absorption solution in the upper portion of the absorption column by passing the gas stream back into this absorption column. This may be effected either via connection of the stripper column by pipework or via direct mounting of the stripper column below the absorber column. This direct coupling may be effected since in accordance with the invention the pressure in the stripping column section and in the absorption column section is the same. Examples of suitable stripping columns include tray columns comprising bubble cap, centrifugal and/or sieve trays, columns comprising structured packings, for example sheet metal packings having a specific surface area of from 100 to 1000 m²/m³ such as Mellapak® 250 Y, and random-packed columns. Also useful, however, are trickle towers and spray towers, and also rotary columns, plate scrubbers, cross-spray scrubbers and rotary scrubbers. Suitable gases are, for example, nitrogen or methane.

The C₄ hydrocarbons-laden absorption medium stream A1′ comprises water. Said water may be separated from the absorption medium A1′ as a stream in a decanter to obtain a stream comprising only the water dissolved in the absorption medium.

The C₄ hydrocarbons-laden absorption medium stream A1′, freed to a very large extent of the water, may be heated up in a heat exchanger and then passed into a desorption column. In one version of the process, the desorption step Dc) is performed by decompressing and/or heating the laden absorption medium. A preferred version of the process utilizes a reboiler in the bottom of the desorption column.

The absorption medium A1 regenerated in the desorption stage may be cooled down in a heat exchanger and recycled into the absorption stage. Low boilers present in the process gas stream, for example ethane or propane, and high-boiling components, such as benzaldehyde, maleic anhydride and phthalic anhydride, may accumulate in the circulation stream. The accumulation may be limited by withdrawing a purge stream. Said stream may be separated into low boilers, regenerated absorbent and high boilers in a distillation column according to the prior art.

The C₄ product gas stream d1 consisting essentially of n-butane, n-butenes and butadiene generally comprises from 20 to 80 vol % of butadiene, from 0 to 80 vol % of n-butane, from 0 to 10 vol % of 1-butene and from 0 to 50 vol % of 2-butenes, where the total amount is 100 vol %. Said stream may further comprise small amounts of isobutane.

Some of the condensed, principally C₄ hydrocarbons-comprising top output from the desorption column is recycled into the top of the column to enhance the separation performance of the column.

In step E) the liquid or gaseous C₄ product streams exiting the condenser may subsequently be separated by extractive distillation with a butadiene-selective solvent into a stream comprising butadiene and the selective solvent and a stream comprising n-butenes.

The extractive distillation may be performed, for example, as described in “Erdöl and Kohle-Erdgas-Petrochemie”, volume 34 (8), pages 343 to 346, or “Ullmanns Enzyklopädie der Technischen Chemie”, volume 9, 4th edition 1975, pages 1 to 18. This comprises contacting the C₄ product gas stream with an extractant, preferably an N-methylpyrrolidone (NMP)/water mixture, in an extraction zone. The extraction zone is generally in the form of a scrubbing column comprising trays, random packings or structured packings as internals. Said column generally comprises from 30 to 70 theoretical plates in order that sufficient separating action is achieved. The scrubbing column preferably comprises a backwashing zone in the top of the column. This backwashing zone is used to recover the extractant present in the gas phase with the aid of a liquid hydrocarbon reflux, for which purpose the top fraction is condensed beforehand. The mass ratio of extractant to C₄ product gas stream in the feed to the extraction zone is generally from 10:1 to 20:1. The extractive distillation is preferably operated at a bottoms temperature in the range of from 100° C. to 250° C., more particularly at a temperature in the range of from 110° C. to 210° C., at an overhead temperature in the range of from 10° C. to 100° C., more particularly in the range of from 20° C. to 70° C., and at a pressure in the range of from 1 to 15 bar, more particularly in the range of from 3 to 8 bar. The extractive distillation column preferably comprises from 5 to 70 theoretical plates.

Suitable extractants are butyrolactone, nitriles such as acetonitrile, propionitrile, methoxypropionitrile, ketones such as acetone, furfural, N-alkyl-substituted lower aliphatic acid amides such as dimethylformamide, diethylformamide, dimethylacetamide, diethylacetamide, N-formylmorpholine, N-alkyl-substituted cyclic acid amides (lactams) such as N-alkylpyrrolidones, in particular N-methylpyrrolidone (NMP). Alkyl-substituted lower aliphatic acid amides or N-alkyl-substituted cyclic acid amides are generally used. Particularly advantageous are dimethylformamide, acetonitrile, furfural and, in particular, NMP.

However, it is also possible to use mixtures of these extractants with one another, for example of NMP and acetonitrile, mixtures of these extractants with co-solvents and/or tert-butyl ethers, for example methyl tert-butyl ether, ethyl tert-butyl ether, propyl tert-butyl ether, n- or isobutyl tert-butyl ether, NMP is particularly suitable, preferably in aqueous solution, preferably comprising from 0 to 20 wt % of water, more preferably comprising from 7 to 10 wt % of water, more particularly comprising 8.3 wt % of water.

The top product stream from the extractive distillation column comprises essentially butane and butenes and small amounts of butadiene and is drawn off in gaseous or liquid form. The stream consisting essentially of n-butane and 2-butene generally comprises up to 100 vol % of n-butane, 0 to 50 vol % of 2-butene, and 0 to 3 vol % of further constituents such as isobutane, isobutene, propane, propene and C₅⁺ hydrocarbons.

The stream consisting essentially of n-butane and 2-butene may be supplied to the C₄ feed of the ODH reactor either wholly or partially. Since the butene isomers in this recycle stream consist essentially of 2-butenes, and 2-butenes are generally oxidatively dehydrogenated to butadiene more slowly than is 1-butene, this recycle stream may be catalytically isomerized before being supplied to the ODH reactor. This makes it possible to adjust the isomer distribution according to the isomer distribution present at thermodynamic equilibrium.

In a step F), the stream comprising butadiene and the selective solvent is distillatively separated into a stream consisting essentially of the selective solvent and a stream comprising butadiene.

The stream obtained at the bottom of the extractive distillation column generally comprises the extractant, water, butadiene and small fractions of butenes and butane and is supplied to a distillation column. Butadiene may be obtained therein as top product or as a side draw. Obtained at the bottom of the distillation column is a stream comprising extractant and possibly water, the composition of the stream comprising extractant and water corresponding to the composition as added to the extraction. The stream comprising extractant and water is preferably returned to the extractive distillation.

When the butadiene is obtained via a side draw the extraction solution thus drawn off is transferred into a desorption zone while the butadiene is once again desorbed and backwashed out of the extraction solution. The desorption zone may, for example, be in the form of a scrubbing column comprising from 2 to 30, preferably from 5 to 20, theoretical plates and optionally a backwash zone comprising, for example, 4 theoretical plates. This backwashing zone is used to recover the extractant present in the gas phase with the aid of a liquid hydrocarbon reflux, for which purpose the top fraction is condensed beforehand. Structured packings, trays or random packings are provided as internals. The distillation is preferably performed at a bottoms temperature in the range of from 100° C. to 300° C., more particularly in the range of from 150° C. to 200° C., and an overhead temperature in the range of from 0° C. to 70° C., more particularly in the range of from 10° C. to 50° C. The pressure in the distillation column is preferably in the range of from 1 to 10 bar. The desorption zone is generally operated at reduced pressure and/or elevated temperature relative to the extraction zone.

The desired product stream obtained at the column top generally comprises from 90 to 100 vol % of butadiene, from 0 to 10 vol % of 2-butene and from 0 to 10 vol % of n-butane and isobutane. Further purification of the butadiene may be accomplished by performing a further prior art distillation.

### EXAMPLE

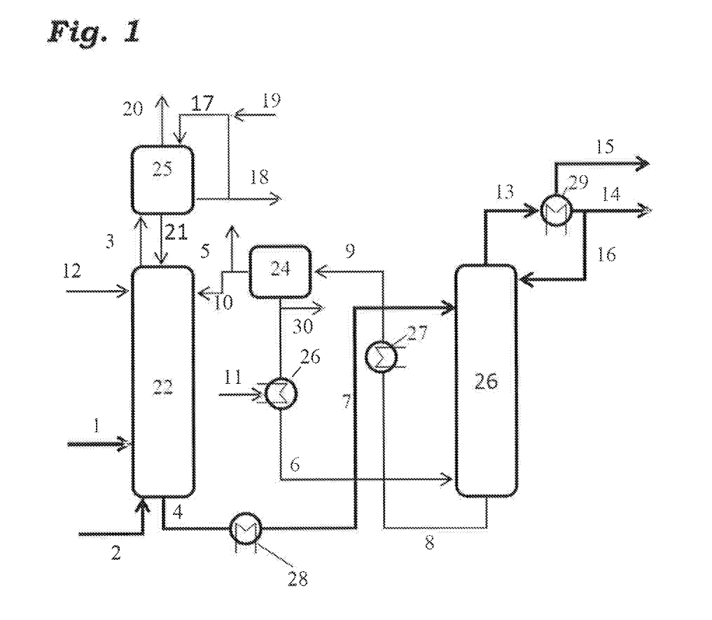

One version of the process according to the invention is shown in FIG. 1.

The process gas mixture exiting the compressor enters stage 30 of the 60 stage absorption column 22 as stream 1 having a temperature of 64° C. and the composition as shown in Table 1. The column top pressure is 10 bar absolute. The column comprises bubble cap trays. In the absorber column the process gas stream flows countercurrently to the unladen absorption medium stream 10 which is supplied from above and consists principally of mesitylene saturated with water. This absorption medium preferentially absorbs the C₄ hydrocarbons and small fractions of the noncondensable gases. The ratio of the mass of the absorption medium stream 10 to the mass of the process gas stream 1 is 2.2:1. The noncondensable gases exit the absorption column principally as stream 3 via the column top and have a temperature of 35° C. and the composition shown in Table 1. The mesitylene concentration in offgas stream 3 is further reduced by passing said stream into a further absorber column 25 and cooling it down further in contact with stream 17. The resulting offgas stream 20 then comprises only 80 mol ppm of mesitylene. It is important in accordance with the invention that condensed-out water be discharged from circuit 17. This may be accomplished via draw 18 or by recycling 21 into absorber column 22. It has proved particularly advantageous when circuit 17 employs the same absorbent as the circuit of absorber column 22 and some of the circuit 17 consisting of absorbent and water is passed as stream 21 into absorber column 22. This withdrawn stream 21 and the fresh absorbent supplied to circuit 17 as stream 19 make it is possible to maintain a maximum water concentration of 42.3 mol %. Circuit 17 thus comprises sufficient organic absorbent having a high peroxide dissolution capacity.

Nitrogen stream 2 desorbs oxygen from the absorption medium stream laden with C₄ hydrocarbons. Absorption medium stream 4 largely freed of oxygen and laden with C₄ hydrocarbons is heated up in heat exchanger 28 and passed as stream 7 into desorber column 26. Here, the C₄ hydrocarbons are removed from the absorption medium stream by stripping vapor stream 6, and exit column 26 as stream 13. Said stream is partially condensed in condenser 29 and gas stream 15 remains. Some of the condensate is recycled into desorber column 26 as stream 16, stream 14 being the C₄ product stream.

The absorption medium and water exit the desorber column 26 as stream 8 and are cooled down in heat exchanger 27 to form stream 9 which is separated in phase separator 24 into an absorption medium stream 10—which is recycled into the absorber column—and aqueous stream 26. A substream 5 may further be withdrawn from the absorption medium stream for the purposes of solvent regeneration. The aqueous stream—from which a further substream 30 may be drawn off—is vaporized in vapor generator 26 to form stream 6. A freshwater stream 11 may further be introduced into steam generator 26.

TABLE

| Stream | 1 | 2 | 3 | 4 | 5 | 6 | 7 | 8 | 9 | 10 | 11 |
|---|---|---|---|---|---|---|---|---|---|---|---|
| Stream type | gas | gas | gas | liquid | liquid | gas | liquid | liquid | liquid | liquid | liquid |
| Temperature | 64.0 | 35.0 | 34.5 | 55.9 | 35.0 | 156.3 | 60.0 | 150.4 | 35.0 | 35.0 | 35.0 |
| Pressure | 10.0 | 10.1 | 10.0 | 10.1 | 10.1 | 5.6 | 5.5 | 5.5 | 10.3 | 10.1 | 10.2 |
| Mass flow rate | 6.8 | 0.21 | 5.8 | 16.5 | 0.10 | 1.7 | 16.5 | 17.0 | 17.0 | 15.2 | 0.12 |

Concentration [wt %]

| | | | | | | | | | | | |
|---|---|---|---|---|---|---|---|---|---|---|---|
| n-Butane | 2.9 | 0.0 | 0.2 | 1.1 | 0.0 | 0.0 | 1.1 | 0.0 | 0.0 | 0.0 | 0.0 |
| Isobutane | 0.0 | 0.0 | 0.0 | 0.0 | 0.0 | 0.0 | 0.0 | 0.0 | 0.0 | 0.0 | 0.0 |
| Isobutene | 0.0 | 0.0 | 0.0 | 0.0 | 0.0 | 0.0 | 0.0 | 0.0 | 0.0 | 0.0 | 0.0 |
| 1-Butene | 0.0 | 0.0 | 0.0 | 0.0 | 0.0 | 0.0 | 0.0 | 0.0 | 0.0 | 0.0 | 0.0 |
| cis-2-Butene | 1.2 | 0.0 | 0.0 | 0.5 | 0.0 | 0.0 | 0.5 | 0.0 | 0.0 | 0.0 | 0.0 |
| trans-2-Butene | 1.5 | 0.0 | 0.0 | 0.6 | 0.0 | 0.0 | 0.6 | 0.0 | 0.0 | 0.0 | 0.0 |
| 1,3-Butadiene | 12.0 | 0.0 | 0.1 | 4.9 | 0.1 | 0.0 | 4.9 | 0.1 | 0.1 | 0.1 | 0.0 |
| Water | 0.0 | 0.0 | 0.2 | 0.0 | 0.0 | 100.0 | 0.0 | 10.1 | 10.1 | 0.0 | 100.0 |
| Mesitylene | 0.8 | 0.0 | 0.2 | 92.6 | 99.9 | 0.0 | 92.6 | 89.8 | 89.8 | 99.9 | 0.0 |
| CO₂ | 1.6 | 0.0 | 1.9 | 0.0 | 0.0 | 0.0 | 0.0 | 0.0 | 0.0 | 0.0 | 0.0 |
| N₂ | 72.9 | 100.0 | 89.1 | 0.1 | 0.0 | 0.0 | 0.1 | 0.0 | 0.0 | 0.0 | 0.0 |
| O₂ | 6.8 | 0.0 | 8.0 | 0.0 | 0.0 | 0.0 | 0.0 | 0.0 | 0.0 | 0.0 | 0.0 |
| CO | 0.3 | 0.0 | 0.4 | 0.0 | 0.0 | 0.0 | 0.0 | 0.0 | 0.0 | 0.0 | 0.0 |

| Stream | 12 | 13 | 14 | 15 | 16 | 17 | 18 | 19 | 20 | 21 |
|---|---|---|---|---|---|---|---|---|---|---|
| Stream type | liquid | gas | liquid | gas | liquid | liquid | liquid | liquid | gas | liquid |
| Temperature | 35.0 | 48.5 | 25.0 | 25.0 | 25.0 | 5.0 | 11.7 | 1.0 | 5.0 | 11.7 |
| Pressure | 10.0 | 5.5 | 5.3 | 5.4 | 5.3 | 10.0 | 10.0 | 10.0 | 10.0 | 10.0 |
| Mass flow rate | 0.02 | 2.47 | 1.14 | 0.07 | 1.25 | 15.00 | 0.02 | 0.05 | 5.74 | 0.05 |

Concentration [wt %]

| | | | | | | | | | | |
|---|---|---|---|---|---|---|---|---|---|---|
| Butane | 0.0 | 15.6 | 15.8 | 10.6 | 15.8 | 0.2 | 0.2 | 0.0 | 0.2 | 0.2 |
| Isobutane | 0.0 | 0.0 | 0.0 | 0.0 | 0.0 | 0.0 | 0.0 | 0.0 | 0.0 | 0.0 |
| Isobutene | 0.0 | 0.1 | 0.1 | 0.1 | 0.1 | 0.0 | 0.0 | 0.0 | 0.0 | 0.0 |
| 1-Butene | 0.0 | 0.0 | 0.0 | 0.0 | 0.0 | 0.0 | 0.0 | 0.0 | 0.0 | 0.0 |
| cis-2-Butene | 0.0 | 6.8 | 6.9 | 3.6 | 6.9 | 0.0 | 0.0 | 0.0 | 0.0 | 0.0 |
| trans-2-Butene | 0.0 | 8.5 | 8.6 | 4.9 | 8.6 | 0.0 | 0.0 | 0.0 | 0.0 | 0.0 |
| 1,3-Butadiene | 0.0 | 67.2 | 67.8 | 47.2 | 67.8 | 0.2 | 0.2 | 0.0 | 0.1 | 0.2 |
| Water | 0.0 | 0.7 | 0.7 | 0.3 | 0.7 | 10.0 | 10.0 | 0.0 | 0.1 | 10.0 |
| TMB | 100.0 | 0.0 | 0.0 | 0.0 | 0.0 | 89.3 | 89.3 | 100.0 | 0.0 | 89.3 |
| CO₂ | 0.0 | 0.1 | 0.0 | 1.3 | 0.0 | 0.1 | 0.1 | 0.0 | 1.9 | 0.1 |
| N₂ | 0.0 | 1.0 | 0.1 | 32.1 | 0.1 | 0.2 | 0.2 | 0.0 | 89.4 | 0.2 |
| O₂ | 0.0 | 0.0 | 0.0 | 0.0 | 0.0 | 0.0 | 0.0 | 0.0 | 8.0 | 0.0 |
| CO | 0.0 | 0.0 | 0.0 | 0.0 | 0.0 | 0.0 | 0.0 | 0.0 | 0.4 | 0.0 |

### LIST OF REFERENCE NUMERALS

1 process gas stream comprising O₂, N₂, C₄, CO, CO₂, H₂O
2 stripping medium, noncondensable gas (e.g. nitrogen, methane or similar gases)
3 offgas stream comprising O₂, N₂, CO, CO₂
4 absorption solution laden with C₄ hydrocarbons, cool
5 unladen absorption solution draw
6 unladen water, vaporized
7 absorption solution laden with C₄ hydrocarbons, heated up
8 unladen absorption solution comprising H₂O, heated up
9 unladen absorption solution comprising H₂O, cool
10 unladen absorption solution
11 fresh water
12 fresh absorption medium
13 C₄ stream, gaseous
14 condensed C₄ stream for extractive distillation
15 gaseous C₄ stream for extractive distillation, still comprises inert gases
16 C₄ stream, liquid reflux
17 circuit around absorber column 25
18 draw from circuit 17
19 fresh absorbent
20 gas stream comprising a smaller amount of absorbent from column 22 than stream 3
21 liquid stream comprising water and absorbent from column 25
22 absorber column with stripping column mounted directly below
23 desorber column
24 Decanter for separating absorber liquid 10 and water 6
25 absorber column for reducing absorption medium concentration in gas stream 3
26 vapor generator
27 heat exchanger
28 heat exchanger
29 condenser
30 water draw

1-13. (canceled)

14. A process for producing butadiene from n-butenes, comprising the steps of:
A) providing an n-butenes—comprising input gas stream a1,
B) feeding the n-butenes—comprising input gas stream a1, an oxygenous gas and an oxygenous cycle gas stream a2 into at least one oxidative dehydrogenation zone and oxidatively dehydrogenating n-butenes to butadiene to obtain a product gas stream b comprising butadiene, unconverted n-butenes, steam, oxygen, low-boiling hydrocarbons, high-boiling secondary components, possibly carbon oxides and possibly inert gases,
Ca) cooling down the product gas stream b and optionally at least partially removing high-boiling secondary components and steam to obtain a product gas stream b′,
Cb) compressing and cooling the product gas stream b′ in at least one compression and cooling stage to obtain at least one aqueous condensate stream c1 and one gas stream c2 comprising butadiene, n-butenes, steam, oxygen, low-boiling hydrocarbons, possibly carbon oxides and possibly inert gases,
Da) absorbing the C₄ hydrocarbons comprising butadiene and n-butenes into an aromatic hydrocarbon solvent absorption medium stream A1 in an absorption column K1 and removing noncondensable and low-boiling gas constituents comprising steam, oxygen, low-boiling hydrocarbons, possibly carbon oxides, aromatic hydrocarbon solvent and possibly inert gases as gas stream d2 from the gas stream c2 to obtain a C₄ hydrocarbons-laden absorption medium stream A1′ and the gas stream d2 and subsequently desorbing the C₄ hydrocarbons from the laden absorption medium stream A1′ to obtain a C₄ product gas stream d1,
Db) at least partially recycling the gas stream d2 into the oxidative dehydrogenation zone as cycle gas stream a2,
wherein said process comprises limiting the content of aromatic hydrocarbon solvent in the cycle gas stream a2 to less than 1 vol % by contacting in a further column K2 the gas stream d2 exiting the removal stage Da) with an at least partially recirculating liquid absorption medium stream A2. for the aromatic hydrocarbon solvent A1, wherein absorption media A1 and A2 are the same aromatic hydrocarbon solvent, and limiting the water content of the liquid absorption medium stream A2 in the column K2 to no more than 80 wt %.

15. The process according to claim 14, wherein said process comprises limiting the water content of the absorption medium stream A2 in the further column K2 to no more than 80 wt % by continually withdrawing a substream of the water-containing absorption medium stream A2 from the further column K2 and replacing it with fresh absorption medium A2 containing no water or less water.

16. The process according to claim 14, wherein said process comprises limiting the water content of the absorption medium stream A2 in the further column K2 to no more than 80 wt % by separating the water-containing absorption medium A2 into an absorption medium phase and a water phase in a phase separator, removing the water phase and reintroducing the absorption medium phase into the further column K2.

17. The process according to claim 16, wherein the phase separator is an integral part of the column bottom of the further column K2.

18. The process according to claim 14, wherein said process comprises limiting the water content of the absorption medium stream A2 in the further column K2 to no more than 80 wt % by passing a substream of the water-containing absorption medium A2 from the column K2 into the absorption column K1.

19. The process according to claim 14, wherein the aromatic hydrocarbon solvent employed as absorption medium A1 in step Da) is selected from the group consisting of toluene, o-, m- or p-xylene, mesitylene, mono-, di- and triethylbenzene and mono-, di- and triisopropylbenzene and mixtures thereof.

20. The process according to claim 19, wherein the aromatic hydrocarbon solvent is mesitylene.

21. The process according to claim 14, wherein the fraction of cycle gas stream a2 is from 10 to 70 vol % based on the sum total of all gas streams fed into the oxidative dehydrogenation zone.

22. The process according to claim 14, wherein step Da) comprises steps Daa) to Dac):
Daa) absorbing the C₄ hydrocarbons comprising butadiene and n-butenes in the aromatic hydrocarbon solvent as absorption medium to obtain a C₄ hydrocarbons-laden absorption medium stream and the gas stream d2,
Dab) removing oxygen from the C₄ hydrocarbons-laden absorption medium stream from step Daa) by stripping with a noncondensable gas stream, and
Dac) desorbing the C₄ hydrocarbons from the laden absorption medium stream to obtain a C₄ product gas stream d1 consisting essentially of C₄ hydrocarbons.

23. The process according to claim 14, wherein said process comprises the further steps:
E) separating the C₄ product stream d1 by extractive distillation with a butadiene-selective solvent into a stream e1 comprising butadiene and the selective solvent and a stream e2 comprising n-butenes;
F) distilling the stream e2 comprising butadiene and the selective solvent to obtain a stream f1 comprising the selective solvent and a stream f2 comprising butadiene.

24. The process according to claim 14, wherein the two columns K1 and K2 are column sections K1 and K2 of a combined column.

25. The process according to claim 24, wherein the combined column comprises a chimney tray between the column sections K1 and K2.

26. The process according to claim 14, wherein said process comprises limiting the water content of the liquid absorption medium stream in the column K2 to no more than 50 wt %.
